(19) United States
(12) Patent Application Publication
(10) Pub. No.: US 2025/0037276 A1
(43) Pub. Date: Jan. 30, 2025

(54) PROCESSING METHOD AND SYSTEM FOR TRACHEAL INTUBATION IMAGES, AND EFFECTIVENESS EVALUATION METHOD FOR TRACHEAL INTUBATION

(71) Applicant: Chia-Chih TSENG, Tainan City (TW)

(72) Inventors: Chia-Chih TSENG, Tainan City (TW); Yu-Hwa WU, Tainan City (TW); Kun-Yi HUANG, Tainan City (TW)

(21) Appl. No.: 18/717,340

(22) PCT Filed: Dec. 10, 2021
(86) PCT No.: PCT/CN2021/137004

§ 371 Date: Jun. 6, 2024

(51) Int. Cl. G06T 7/00

(52) U.S. Cl. CPC G06T 7/0012; G06T 2207/30004

(57) ABSTRACT

A processing method and system for tracheal intubation images, and an effectiveness evaluation method for tracheal intubation are provided. A database containing images of the target structure and important steps in the tracheal intubation process is established first, and then the machine learning based on the images stored in the database is performed to construct a system that automatically recognizes the target structure. Wherein, the time difference between the two objects recognized in the intubation image is defined as a stage intubation time, and the stage intubation times are tailored in chronological order to establish the intubation time sequence. The intubation time sequence and stage intubation time are used as methods for evaluating the effectiveness of tracheal intubation and establishing a staged effectiveness evaluation model for tracheal intubation.

### BACKGROUND

### Technology Field

The present invention relates to a processing method and system and, in particular, to a processing method and system for tracheal intubation images, and an effectiveness evaluation method for tracheal intubation.

### Description of Related Art

Tracheal intubation is a common high-risk, high-tech medical behavior. Moreover, tracheal intubation must be completed in a very short time period. Failure to complete tracheal intubation within a few minutes may cause damages of important organs. Limited by intubation tools and techniques, the current evaluation of difficulty in intubation is only based on the operator's subjective judgment and clinical intubation results (e.g. time for intubation, failure intubations, etc.), or based on specific image recognition, resulting in considerable differences in the evaluation of difficulty in intubation. Therefore, it is impossible to conduct integration and communication on research, and there is no objective evaluation method to evaluate the results and qualifications of this skill in clinical teaching training that all doctors should have.

In recent years, the diversification and clinical use of image-assisted intubation tools has become popular. However, based on clinical practices, it is found that the evaluation of intubation difficulty and intubation methods used in the past cannot be directly repeated and applied to the technology of image-assisted intubation. The conventional art uses video laryngoscope or video stylet to take simultaneous photography during the intubation process, but the images recorded by these practices are only for subsequent viewing and learning, or analysis the differences of local structures. There is still no systematic structural analysis and time sequence analysis of the images of the intubation process, and the images of the intubation process are never be used in the analysis of intubation difficulty and the evaluation of the results of intubation teaching trainings.

### SUMMARY

The present invention provides a processing method and system for tracheal intubation images based on the clinical operations and training needs of high-risk, high-tech tracheal intubation, which can provide real-time segmentation of the entire image during staged intubation, serialized processing and analysis of integrated time period, and then establish an effectiveness evaluation method for tracheal intubation, thereby solving the current problem of subjective judgment and manual interpretation that is difficult to immediately assist operation and training feedback.

To achieve the above objective, a processing method for tracheal intubation images of this invention includes: establishing a database containing a plurality of structural objects, wherein the database stores at least one first intubation process image, and the structural objects are defined by the at least one first intubation process image; performing an object recognition of a second intubation process image based on the defined structural objects, so as to obtain multiple target objects in the second intubation process image identical to the structural objects; and determining multiple time points of recognizing the multiple target objects in the second intubation process image so as to obtain stage intubation times and intubation time sequences of the target objects. Wherein, the multiple time points of recognizing the multiple target objects in the second intubation process image define n time points, a time difference between any two of the time points of recognizing the target objects is defined as one of the stage intubation times, and a plurality of the stage intubation times are tailored in chronological order to establish the intubation time sequences of the target objects in the second intubation process image.

In one embodiment, the first intubation process image with an intubation difficulty scale of zero is objected to analysis and model establishment so as to define the structural objects.

In one embodiment, the structural objects are selected from a group consisting of lip, epiglottis, laryngopharynx, glottis, endotracheal tube, and endotracheal tube marked black line.

In one embodiment, the processing method further includes: determining multiple time points of the structural objects appearing in the first intubation process image so as to obtain stage intubation times and intubation time sequences of the structural objects; wherein the multiple time points of the structural objects appearing in the first intubation process image define n time points, a time difference between any two of the time points of the structural objects appearing in the first intubation process image is defined as one of the stage intubation times, and a plurality of the stage intubation times are tailored in chronological order to establish the intubation time sequences of the structural objects in the first intubation process image; and drawing an intubation time sequence diagram and an intubation ability-time sequence analysis diagram of the first intubation process image based on the stage intubation times and the intubation time sequences of the structural objects.

In one embodiment, the processing method further includes: drawing an intubation time sequence diagram and an intubation ability-time sequence analysis diagram of the second intubation process image based on the stage intubation times and the intubation time sequences of the target objects.

To achieve the above objective, a processing system for tracheal intubation images of the invention includes a database and an electronic device. The database stores at least one first intubation process image, and the at least one first intubation process image defines a plurality of structural objects. The electronic device is electrically connected to the database. The electronic device includes one or more processing units and a memory unit. The one or more processing units are electrically connected to the memory unit, and the memory unit stores one or more instructions. The one or more processing units perform, when executing the one or more instructions, following steps: performing an object recognition of a second intubation process image based on the defined structural objects, so as to obtain multiple target objects in the second intubation process image identical to the structural objects; and determining multiple time points of recognizing the multiple target objects in the second intubation process image so as to obtain stage intubation times and intubation time sequences of the target objects; wherein the multiple time points of recognizing the multiple target objects in the second intubation process image define n time points, a time difference between any two of the time points of recognizing the target objects is defined as one of the stage intubation times, and a plurality of the stage intubation times are tailored in chronological order to establish the intubation time sequences of the target objects in the second intubation process image.

In one embodiment, the database is stored in the memory unit or a cloud device.

In one embodiment, the one or more processing units further perform following steps: determining multiple time points of the structural objects appearing in the first intubation process image so as to obtain stage intubation times and intubation time sequences of the structural objects; wherein the multiple time points of the structural objects appearing in the first intubation process image define n time points, a time difference between any two of the time points of the structural objects appearing in the first intubation process image is defined as one of the stage intubation times, and a plurality of the stage intubation times are tailored in chronological order to establish the intubation time sequences of the structural objects in the first intubation process image; and drawing an intubation time sequence diagram and an intubation ability-time sequence analysis diagram of the first intubation process image based on the stage intubation times and the intubation time sequences of the structural objects.

In one embodiment, the one or more processing units further perform following step: drawing an intubation time sequence diagram and an intubation ability-time sequence analysis diagram of the second intubation process image based on the stage intubation times and the intubation time sequences of the target objects.

To achieve the above objective, an effectiveness evaluation method for tracheal intubation of this invention includes: the above-mentioned processing method; and performing an effectiveness evaluation of tracheal intubation with the second intubation process image based on the stage intubation times and the intubation time sequences of the target objects and the structural objects.

In one embodiment, the effectiveness evaluation of tracheal intubation comprises: comparing the stage intubation times, the intubation time sequence diagrams and the intubation ability-time sequence analysis diagrams of the first intubation process image and the second intubation process image so as to evaluate intubation effectiveness of different stages in the second intubation process image.

As mentioned above, the processing method for tracheal intubation images of this invention includes: establishing a database containing a plurality of structural objects, wherein the database stores at least one first intubation process image, and the structural objects are defined by the at least one first intubation process image; performing an object recognition of a second intubation process image based on the defined structural objects, so as to obtain multiple target objects in the second intubation process image identical to the structural objects; and determining multiple time points of recognizing the multiple target objects in the second intubation process image so as to obtain stage intubation times and intubation time sequences of the target objects; wherein the multiple time points of recognizing the multiple target objects in the second intubation process image define n time points, a time difference between any two of the time points of recognizing the target objects is defined as one of the stage intubation times, and a plurality of the stage intubation times are tailored in chronological order to establish the intubation time sequences of the target objects in the second intubation process image. Therefore, based on the clinical operations and training needs of high-risk, high-tech tracheal intubation, the proposed processing method and system for tracheal intubation images of this invention can provide the stage intubation time and intubation time sequence of the entire tracheal intubation image, thereby providing the real-time segmentation of the entire image during staged intubation, serialized processing and analysis of integrated time period, and then establish an effectiveness evaluation for tracheal intubation. As a result, the invention can solve the current problem of subjective judgment and manual interpretation that is difficult to immediately assist operation and training feedback.

### BRIEF DESCRIPTION OF THE DRAWINGS

FIG. 1A is functional block diagram showing the processing system for tracheal intubation images according to an embodiment of this invention.

FIG. 1B is a flow chart showing the steps of the processing method for tracheal intubation images according to an embodiment of this invention.

FIG. 2 is another flow chart showing the steps of the processing method for tracheal intubation images of this invention.

FIG. 3A and FIG. 3B are respectively an intubation time sequence diagram and an intubation ability-time sequence analysis diagram of the first intubation process image according to an embodiment of this invention.

FIG. 4A and FIG. 4B are respectively an intubation time sequence diagram and an intubation ability-time sequence analysis diagram of the second intubation process image according to an embodiment of this invention.

FIG. 5 is a flow chart showing the steps of the effectiveness evaluation method for tracheal intubation according to an embodiment of this invention.

### DETAILED DESCRIPTION OF THE DISCLOSURE

The processing method and system for tracheal intubation images, and the effectiveness evaluation method for tracheal intubation according to embodiments of the present invention will be described hereinafter with reference to the relevant drawings, wherein the same elements will be described with the same reference numerals.

The cases for tracheal intubation analysis included in this disclosure were collected in accordance with the principles approved by the Human Experimentation Committee (IRB NCKUH B-ER-107-088). In addition, the processing system presented in this disclosure can also be called an analysis system, and the processing method can also be called an analysis method. Moreover, the first intubation process images and the second intubation process images that appear in this disclosure are just for distinction, and they are both entire images recorded during the tracheal intubation process.

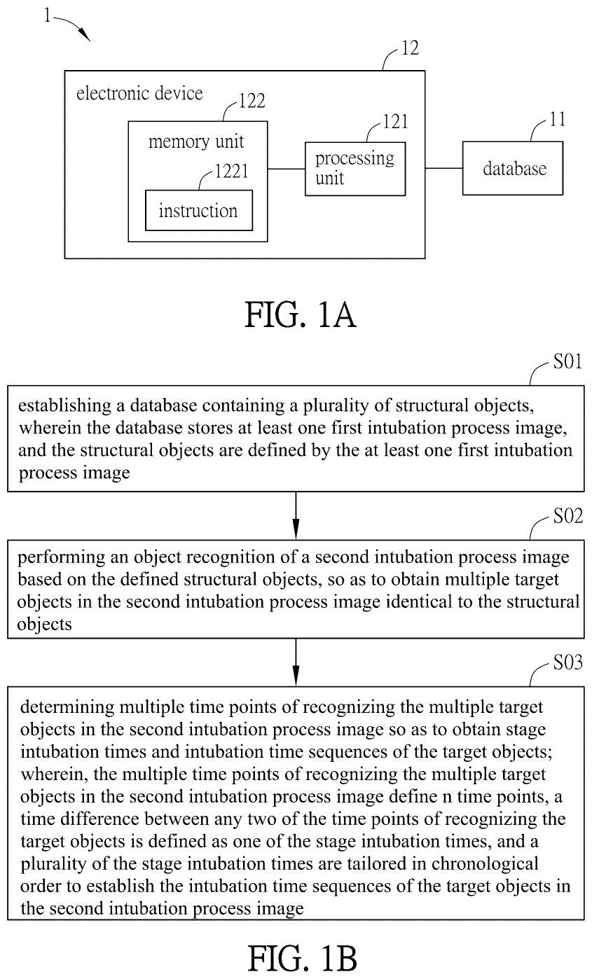

FIG. 1A is functional block diagram showing the processing system for tracheal intubation images according to an embodiment of this invention, and FIG. 1B is a flow chart showing the steps of the processing method for tracheal intubation images according to an embodiment of this invention.

Referring to FIG. 1A and FIG. 1B, the processing system for tracheal intubation images 1 includes a database 11 and an electronic device 12.

The database 11 can store at least one first intubation process image, and a plurality of structural objects can be defined from the at least one first intubation process image. Specifically, in order to conduct analysis and processing procedures to establish an evaluation model and standard for the effectiveness of tracheal intubations, it is necessary to first establish a database 11 containing multiple structural objects, and these structural objects are defined based on the at least one first intubation process image. Preferably, these structural objects are defined based on a plurality of first intubation process images.

In this embodiment, once the patient is expected to undergo a general anesthesia operation with tracheal intubation, he or she must sign the operation agreement first. The entire image recorded during the intubation process will be collected in the database 11, and the information used for analysis is selected from the database 11 and includes cases of successful intubation made by the attending physician of the anesthesiology department. All patients were intubated with, for example but not limited to Trachway blade, and the entire intubation process was recorded with a camera provided at the front end of the blade. After the intubation, the intubation difficulty scale (IDS) analysis and intubation time evaluation are performed to establish image recognition of the basic structure or characteristics of successful intubation, while integrating and analyzing variations in process intervals and automating analysis and evaluation.

In the database 11 of this embodiment, the first intubation process image with an intubation difficulty scale of zero (i.e. low-difficult intubation case) is objected to analysis and model establishment so as to define the structural objects. The structural objects are selected from, for example but not limited to, a group consisting of lip, epiglottis, laryngopharynx, glottis, endotracheal tube, and endotracheal tube marked black line(s) (e.g. two circular black lines in the endotracheal tube). This invention is not limited thereto. In this embodiment, there are a total of 6 structural objects, including the lip, epiglottis, laryngopharynx, glottis, endotracheal tube and endotracheal tube marked black lines, but this invention is not limited thereto. In different embodiments, the structural objects defined based on the tracheal intubation images can be different, and the number of the structural objects can be greater or less than 6. In practice, users can define proper structural objects with a number and structures different from the above embodiment based on the intubation process images according to the needs of effectiveness evaluation.

In some embodiments, for example, 33 entire image cases of intubation process can be selected from the database 11 that stores the first intubation process images, and screened by multiple senior specialists to deconstruct the intubation process into different stages according to time sequences and to mark and confirm the defined structural objects in the images. For example, the total screened images include 27 images of lip, 173 images of epiglottis, 366 images of laryngopharynx, 377 images of glottis, 24 images of endotracheal tube, and 345 images of endotracheal tube marked black line (including images of 6 kinds of structural objects appearing in time sequence). These structural object images can be used to train the object recognition model based on YOLOv3 (Real-Time Object Detection), which can be used for object recognition in other intubation images later. In some embodiments, the file of the first intubation process image can be divided into 30 frames (30 fps), and then the image object recognition (Object Detection) can be performed. In this case, any two objects can be combined into one stage intubation time, so intubation images can be trimmed into intubation time sequences, thereby analyzing the timing of each structural object in the tracheal intubation process.

In other words, the database 11 containing images of target structures and important steps in the tracheal intubation process is first established, and the artificial intelligence (AI) is simultaneously used to learn based on the images stored in the database 11 to construct an AI recognition system that can automatically recognize the above-mentioned related structures. In this disclosure, the images of the entire intubation process are deconstructed in stages according to the structural objects recognized based on time, thereby recognizing the corresponding time points and operational meanings of each structural object shown in the image sequence, and then obtaining the stage intubation times and the intubation time sequence generated by tailoring the stage intubation times in chronological order. For example, assuming that the intubation process image can define n structural objects (n≥2) according to the time sequence, and these n structural objects respectively correspond to n different time points in the image, then the difference between the time points of any two adjacent or non-adjacent structural objects can be defined as one stage intubation time.

Taking n=6 as an example, since any two structural objects can define one stage intubation time, the entire intubation process can be deconstructed into at least 5 intubation stages corresponding to 5 (6−1=5) stage intubation times in chronological order, and can be deconstructed into up to 15 intubation stages corresponding to 15 (6*5/2=15) stage intubation times in chronological order. Each intubation stage has its own time and operational meanings. Then, the stage intubation times are tailored in chronological order to obtain the intubation time sequences of the entire intubation process. In this embodiment, when n=6, the structural objects in order of appearance in time (i.e., the first structural object to the sixth structural object) can deconstruct at least 5 intubation stages corresponding to the 5 stage intubation times (e.g. t1 to t5). Specifically, the stage intubation time t1 corresponds to the period between the first structural object and the second structural object, the stage intubation time t2 corresponds to the period between the second structural object and the third structural object, the stage intubation time t3 corresponds to the period between the third structural object and the fourth structural object, the stage intubation time t4 corresponds to the period between the fourth structural object and the fifth structural object, and the stage intubation time t5 corresponds to the period between the fifth structural object and the sixth structural object. Therefore, there are a total of 5 intubation stages. The 5 stage intubation times (t1, t2, t3, t4 and t5) can be tailored in chronological order to obtain the intubation time sequence of the 6 structural objects. In another case, for example, three stage intubation times including (t1+t2), t3 and (t4+t5) can be tailored in chronological order to obtain the intubation time sequence of the 6 structural objects, wherein these three stage intubation times respectively correspond to the period between the first structural object and the third structural object, the period between the third structural object and the fourth structural object, and the period between the fourth structural object and the sixth structural object. In different embodiments, if n=8, the entire intubation process can be deconstructed into at least 7 intubation stages corresponding to 7 (8−1=7) stage intubation times, and can be deconstructed into up to 28 intubation stages corresponding to 28 (8*7/2=28) stage intubation times. Then, some of the stage intubation times can be tailored in chronological order to obtain the intubation time sequences of the entire intubation process, and so on.

In this embodiment, an image annotation tool, LabelImg marking software, is used as the annotation tool for the objects. According to the structures and characteristics of the objects, an expert conference can be held to select the structural objects, to assign definition principle to each structural object, and to test verification and correction, and then the above-mentioned 6 structural objects can be defined. Then, these 6 structural objects can be used to train artificial intelligence. After repeated verification and correction, the objects of other subsequent intubation images can be automatically recognized by the AI system, and the accuracy can almost reach perfect, thereby achieving automatic effectiveness evaluation of tracheal intubation images.

In order to improve the accuracy of AI recognition to be perfect, the AI must be repeatedly verified and corrected to verify the accuracy of the object recognition. In this disclosure, in addition to providing the multiple first intubation process images, which are used to define the structural objects, to the AI system for testing (verifying), other intubation process images (either IDS=0 or IDS≠0 is acceptable) can be provided for testing (verifying), and continuously train and correct the AI recognition ability. Therefore, the AI system can achieve perfect recognition accuracy of target objects.

In addition, it is worth noting that during the process of tracheal intubation, the temporal significance of the intubation images of each anatomical structure is defined as follows. The intubation image of lip indicates the starting point of the intubation process, wherein the disappearance of the lip means that the camera has entered the oral cavity. The intubation image of epiglottis indicates that the camera has entered the oral cavity, passed through the tongue, and correctly reached the base of the tongue. For beginners, the intubation image of epiglottis means that the blade has been slid into the base of the tongue, and the basic operation is correct while the intubation process does not deviate too much from the midline, wherein the blade in the center will affect the movement range of the epiglottis. The intubation image of glottis indicates that, after seeing the epiglottis, the position, strength and angle of the blade can be adjusted to get the best view of glottis opening. The intubation image of larynx superior view indicates that whether the glottis can be quickly seen in the larynx superior view is an indicator of difficult intubation in the minds of anesthesiologists (however, the past intubation images cannot specifically mark this time point). The intubation image of arytenoid commissure (AC) indicates that the earliest throat structure exposed during intubation. The shape of arytenoid commissure is different from the opening of esophagus, so it is an important anatomical structure for distinguishing the openings of esophagus and trachea. The intubation image of the front end of endotracheal tube indicates that the endotracheal tube has been appeared in the field of view, which means that the intubation tube can be delivered from the opening to the throat. The intubation image of the middle part of the endotracheal tube indicates that the intubation tube has aimed at the throat and it confirms that the intubation tube is successfully controlled to reach the larynx. The intubation image of the endotracheal tube marked black line indicates that the intubation tube has been positioned and the intubation process has been completed if the marked black line disappears.

Referring again to FIG. 1A, the electronic device 12 is electrically connected to the database 11. The electronic device 12 can be a computer, a server, a mobile phone or a tablet, and this invention is not limited thereto. In some embodiments, the electrical connection between the electronic device 12 and the database 11 may be a wireless connection or a wired connection. The wireless connection may be established through a Wi-Fi module, a Bluetooth module or a mobile network (3G, 4G or 5G), so that the electronic device 12 can receive, store and process the data stored in the database 11. The electronic device 12 may include one or more processing units 121 and a memory unit 122, and the one or more processing units 121 are electrically connected to the memory unit 122. For example, in the embodiment as shown in FIG. 1A, the electronic device 12 includes a processing unit 121 and a memory unit 122, and the aforementioned database 11 can be located in the memory unit 122 or a cloud device. In another embodiment, the database 11 can be located in an independent computer-readable storage medium (for example but not limited to solid state drive, USB memory, or any type of memory) or a memory chip. When the database 11 is located in a cloud device, the electronic device 12 needs to download the data stored in the database 11 from the cloud device to the memory unit 122 before performing further process and analysis steps. Then, the processing unit 121 performs the processing and analysis steps. If the database 11 is located in the memory unit 122, the downloading step is not needed. When the database 11 is located in an independent computer-readable storage medium, the processing unit 121 can read in the content stored in the computer-readable storage medium as long as the computer-readable storage medium is plugged in the electronic device 12.

The processing unit 121 can access the data stored in the memory unit 122 and can include the core control components of the electronic device 12, such as at least one CPU and memory, or other control hardware, software or firmware. In addition, the memory unit 122 can be a non-transitory computer readable storage medium, which may include, for example but not limited to, at least one memory, memory card, memory chip, optical disc, video tape, computer tape, or any combination thereof. In some embodiments, the aforementioned memory may include read-only memory (ROM), flash memory, field-programmable gate array (FPGA), or solid state disk (SSD), or other kinds of memory, or a combination thereof.

The memory unit 122 may store at least one application software, which may include one or more program instructions 1221. After the above-mentioned database 11 is established, and when the one or more program instructions 1221 of the application software stored in the memory unit 122 are executed by the one or more processing units 121, the one or more processing units 121 may at least perform the following steps: performing an object recognition of a second intubation process image based on the defined structural objects, so as to obtain multiple target objects in the second intubation process image identical to the structural objects (step S02 of FIG. 1B); and determining multiple time points of recognizing the multiple target objects in the second intubation process image so as to obtain stage intubation times and intubation time sequences of the target objects; wherein the multiple time points of recognizing the multiple target objects in the second intubation process image define n time points, a time difference between any two of the time points of recognizing the target objects is defined as one of the stage intubation times (equal to or greater than (n−1) of stage intubation times can be obtained based on the second intubation process image), and a plurality of the stage intubation times are tailored in chronological order to establish the intubation time sequences of the target objects in the second intubation process image (step S03 of FIG. 1B).

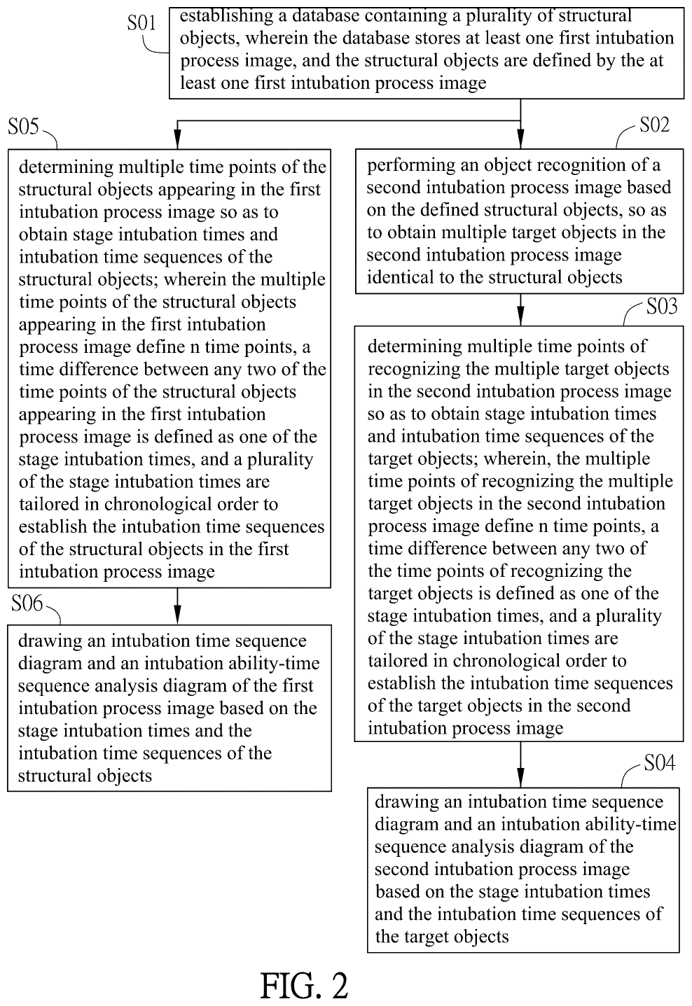

In addition, the one or more processing units 121 may further perform the following step: drawing an intubation time sequence diagram and an intubation ability-time sequence analysis diagram of the second intubation process image based on the stage intubation times and the intubation time sequences of the target objects (step S04 of FIG. 2). Moreover, the one or more processing units 121 may further perform the following steps: determining multiple time points of the structural objects appearing in the first intubation process image so as to obtain stage intubation times and intubation time sequences of the structural objects; wherein the multiple time points of the structural objects appearing in the first intubation process image define n time points, a time difference between any two of the time points of the structural objects appearing in the first intubation process image is defined as one of the stage intubation times (equal to or greater than (n−1) of stage intubation times can be obtained based on the first intubation process image), and a plurality of the stage intubation times are tailored in chronological order to establish the intubation time sequences of the structural objects in the first intubation process image (step S05 of FIG. 2); and drawing an intubation time sequence diagram and an intubation ability-time sequence analysis diagram of the first intubation process image based on the stage intubation times and the intubation time sequences of the structural objects (step S06 of FIG. 2).

The above steps S02 to S06 will be described in detail below.

As shown in FIG. 1B, the processing method for tracheal intubation images of the present invention may include steps S01 to S03.

The step S01 is to establish a database 11 containing a plurality of structural objects, wherein the database 11 stores at least one first intubation process image, and the structural objects are defined by the at least one first intubation process image. As previously mentioned, the first step is to determine and define the structural objects of at least one first intubation process image (preferably multiple first intubation process images) to establish a subsequent recognition references. In this embodiment, multiple first intubation process images with an intubation difficulty scale of zero are used to analyze and establish model(s) to define these structural objects. In addition, these structural objects in this embodiment appearing in time sequence include, for example but not limited to, the above-mentioned lip, epiglottis, laryngopharynx, glottis, endotracheal tube and endotracheal tube marked black lines, a total of 6 target structures (but not limited to 6). These six structural objects, the first intubation process images, the stage intubation times, and the intubation time sequences can all be stored in the database 11.

The step S02 is to perform an object recognition of a second intubation process image based on the defined structural objects, so as to obtain multiple target objects in the second intubation process image identical to the structural objects. In this case, the second intubation process image and the recognized target objects may also be stored in the database 11. Specifically, in order to evaluate the intubation effectiveness of the subsequent intubation images (i.e., the second intubation process images), it is necessary to first recognize the objects in the second intubation process images during the intubation process, and the target objects recognized from the second intubation process images must be identical to the structural objects in the first intubation process images, so that the effectiveness of each stage in the intubation process can be evaluated on the same basis. In this disclosure, the stages can each represent different operational definitions and meanings, and can be independently evaluated or improved. In some embodiments, the AI system trained by the above steps can be used to perform object recognition on the subsequent intubation process images, and then multiple target objects having structures identical to those of the obtained structural objects can be obtained. Herein, 6 or more of the target objects can be obtained, wherein the number of objects must be the same as the number of stages for performing the comparison. In this embodiment, the second intubation process images are, for example, tracheal intubation process images obtained when other doctors (such as but not limited to PGY interns) learn or practice the actual operation of tracheal intubation in the anesthesia department.

The step S03 is to determine multiple time points of recognizing the multiple target objects in the second intubation process image so as to obtain stage intubation times and intubation time sequences of the target objects; wherein, the multiple time points of recognizing the multiple target objects in the second intubation process image define n time points, a time difference between any two of the time points of recognizing the target objects is defined as one of the stage intubation times, and a plurality of the stage intubation times are tailored in chronological order to establish the intubation time sequences of the target objects in the second intubation process image. Since the second intubation process image is also a time sequence image of the intubation process, the time points that various target objects (such as but not limited to lip, epiglottis, laryngopharynx, glottis, endotracheal tube, and endotracheal tube marked black lines, etc.) appear sequentially in the second intubation process image can be obtained, as mentioned above. Then, the stage intubation times of these target objects can be obtained, and the intubation time sequences can also be obtained by tailoring the stage intubation times in chronological order. The obtained stage intubation times and intubation time sequences can be used for subsequent evaluation.

In this embodiment, the time points at which these target objects appear sequentially in the second intubation process image can define, for example, 6 time points. The time difference between any two target objects can be defined as one stage intubation time. At least 5 stage intubation timed (corresponding to 5 stages) and at most 15 stage intubation timed (corresponding to 15 stages) can be obtained based on the second intubation process image, and these stage intubation times (and intubation stage) can be tailored in chronological order of the appearances of the target objects so as to establish the intubation time sequences of the second intubation process image. In other words, assume that the time points at which 6 (n=6) target objects, including the lip, epiglottis, laryngopharynx, glottis, endotracheal tube, and endotracheal tube marked black line, appear sequentially in the image are t1, t2, . . . , t6, then (t2−t1) indicates the stage intubation time of the stage from lip to epiglottis, (t3−t2) indicates the stage intubation time of the stage from epiglottis to laryngopharynx, . . . , and (t6−t5) indicates the stage intubation time of the stage from endotracheal tube to endotracheal tube marked black line. In addition, (t3−t1) indicates the stage intubation time of the stage from lip to laryngopharynx, (t5−t2) indicates the stage intubation time of the stage from epiglottis to endotracheal tube, (t6−t1) indicates the stage intubation time of the stage from lip to endotracheal tube marked black line, and so on. Therefore, a total of stage intubation times equal to or greater than 5 stages (up to 15 stages in this embodiment) can be obtained.

For example, if the difference between the time point of first glottis image (Glottis 1st) and the time point of first epiglottis image (Epiglottis 1st) is 7 seconds, it means that the time spent in the stage from epiglottis to glottis is 7 seconds (i.e., this stage intubation time is 7 seconds), and so on. It is worth noting that the time difference between two target objects (or two structural objects) is not limited to two adjacent target objects (or two adjacent structural objects) in the time sequence, and the stage intubation time can be calculated based on two non-adjacent target objects (or two non-adjacent structural objects) so as to obtain the stage intubation time corresponding to the intubation stage and then to evaluate the effectiveness of the intubation stage. For example, it can be performed separately or simultaneously to calculate the time difference (t6−t4) between the time point of appearing the (non-adjacent) glottis (time point t4) and the time point of disappearance of the endotracheal tube marked black line (time point t6), so as to obtain the stage intubation time of this intubation stage. (t6−t4), and then to evaluate the effectiveness of this intubation stage. The same method can be used for other intubation stages.

FIG. 2 is another flow chart showing the steps of the processing method for tracheal intubation images of this invention. Referring to FIG. 2, in addition to the above-mentioned steps S01 to S03, the processing method may further include steps S04 to S06. In this disclosure, the time sequence of the step S04 and the step S05 (as well as the step S06) is not limited. For example, the step S04 can be performed before the step S05 (and the step S06); or the step S05 (and the step S06) can be performed before the step S04; or the step S04 and the step S05 (as well as the step S06) are performed simultaneously.

The step S05 and step S06 will be described first, and then the step S04 will be described. In this embodiment, the step S05 is to determine multiple time points of the structural objects appearing in the first intubation process image so as to obtain stage intubation times and intubation time sequences of the structural objects; wherein the multiple time points of the structural objects appearing in the first intubation process image define n time points, a time difference between any two of the time points of the structural objects appearing in the first intubation process image is defined as one of the stage intubation times, and a plurality of the stage intubation times are tailored in chronological order to establish the intubation time sequences of the structural objects in the first intubation process image. Similarly, since the first intubation process image is also a time sequence image of the intubation process, the process of obtaining, for example, 6 time points and 5 or more (e.g. up to 15) of the stage intubation times corresponding to the appearances of the structural objects in the first intubation process image can be obtained by the method as the above-mentioned step S03, which is used to obtain the stage intubation times and intubation time sequences of the target objects in the second intubation process image. Then, the obtained stage intubation times can be tailored in chronological order to establish the intubation time sequences of the structural objects in the first intubation process image.

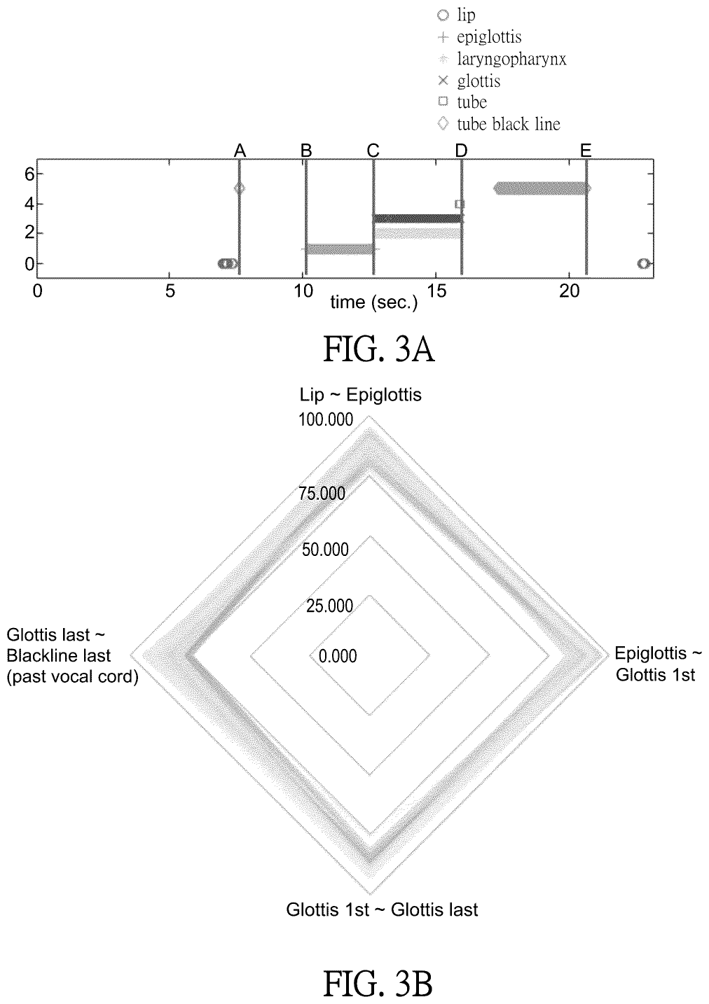

The step S06 is to draw an intubation time sequence diagram and an intubation ability-time sequence analysis diagram of the first intubation process image based on the stage intubation times and the intubation time sequences of the structural objects. In this embodiment, based on the (first) intubation image file with IDS equal to 0 in the above-mentioned database 11, three senior anesthesiology doctors jointly marked the above-mentioned 6 structural objects and their corresponding time points of appearances, which can be used to establish the intubation time sequences of multiple stage intubations and standard airway intubation throughout the whole process for the subsequent comparison of effectiveness. The results can refer to the intubation time sequence diagram of the first intubation process image as shown in FIG. 3A, and the intubation ability-time sequence analysis diagram of the first intubation process image as shown in FIG. 3B.

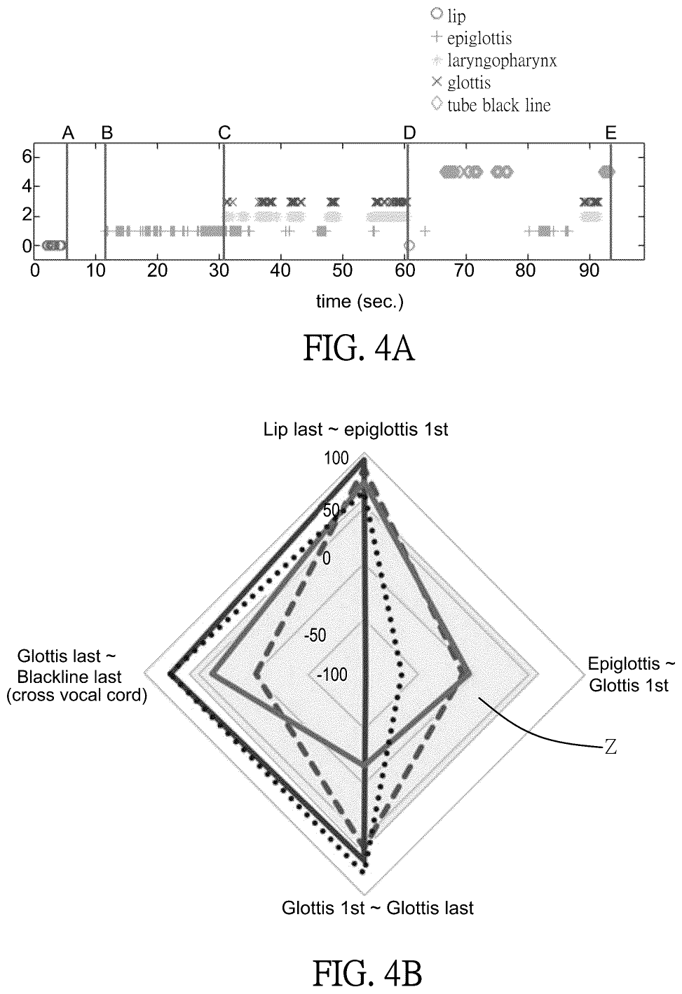

In addition, the step S04 is to draw an intubation time sequence diagram and an intubation ability-time sequence analysis diagram of the second intubation process image based on the stage intubation times and the intubation time sequences of the target objects. Similarly, based on the 6 target objects recognized in the second intubation process images, which are collected from the tracheal intubations performed by the PGY interns, and their corresponding intubation time sequences (step S03), the stage intubation times and intubation time sequence diagram of the second (novice) intubation process images can be drawn as shown in FIG. 4A, and the intubation ability-time sequence analysis diagram of the second (novice) intubation process image can be drawn as shown in FIG. 4B.

As shown in FIG. 3A and FIG. 4A, the abscissa is time (seconds), A indicates the last lip image (Lip last), B indicates the first epiglottis image (Epiglottis 1st), C indicates the first larynx image (larynx 1st), and D indicates the last glottis image without tube (Last free glottis), E indicates the last black line image (Last blackline). The time spent in each stage of different tracheal intubation images can be obtained from FIG. 3A and FIG. 4A. To be noted, not all the objects (and its corresponding time points) need to appear in the intubation time sequence diagram, and one or some objects may not appear in the intubation time sequence diagram depending on the user's evaluation method.

In the embodiments of FIG. 3A and FIG. 4A, there are 6 objects but only 5 time points and 4 intubation stages are shown (the 4 stage intubation times are: a stage from A to B, a stage from B to C, a stage from C to D, and a stage from D to E). The stage can each represent different operational definitions and meanings, and can be independently evaluated or improved. For example, only a certain intubation stage or all of the intubation stages can be evaluated individually. In different embodiments, two adjacent objects of the 6 objects (or n objects) can be calculated separately so as to obtain 5 intubation stages (5 stage intubation times). Alternatively, it can be performed according to the user's needs to generate more than 5 stage intubation times, and then evaluate the stage intubation time between one target object and another target object (such as the stage from A to B, the stage from B to C, the stage from C to D, the stage from D to E, or the integrated stage from B to D, the integrated stage from A to D, and the likes) and their effectiveness. Alternatively, the operational meanings of all stages of intubation can be separately deconstructed, and the effectiveness of each stage can be separately evaluated, or the effectiveness of only one certain intubation stage can be evaluated. This invention is not limited thereto.

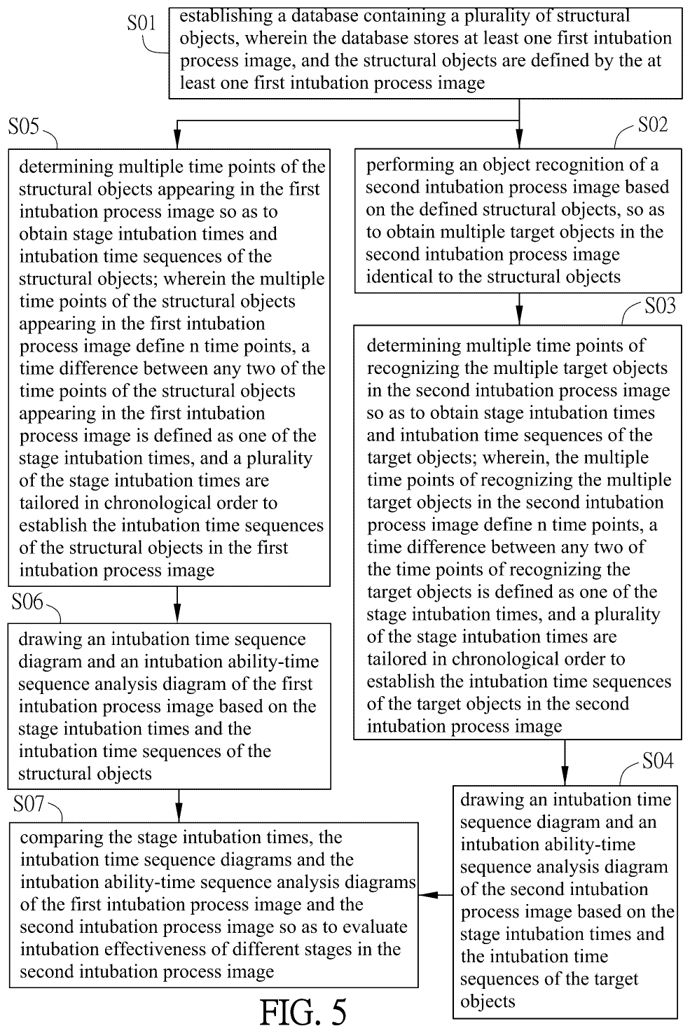

FIG. 5 is a flow chart showing the steps of the effectiveness evaluation method for tracheal intubation according to an embodiment of this invention.

The present invention also proposes an effectiveness evaluation method for tracheal intubation, which can be applied to the above-mentioned processing system 1 and method for tracheal intubation images. The processing system 1 for tracheal intubation images 1 and the processing method for tracheal intubation images have been described in detail above and will not be described further here. The effectiveness evaluation method for tracheal intubation of the present invention can be used to automatically evaluate tracheal intubations, and can include the above-mentioned processing methods (or steps) of tracheal intubation and the effectiveness evaluation steps.

As shown in FIG. 5, the processing procedure (or method) of tracheal intubation include steps S01 to S06. The steps S01 to S06 have been described in detail above and will not be described in detail here. In addition, the effectiveness evaluation step is to perform an effectiveness evaluation of tracheal intubation with the second intubation process image based on the intubation time sequences of the target objects and the structural objects. In this disclosure, as shown in FIG. 5, the effectiveness evaluation of tracheal intubation with the second intubation process image includes step S07: comparing the stage intubation times, the intubation time sequence diagrams and the intubation ability-time sequence analysis diagrams of the first intubation process image and the second intubation process image so as to evaluate intubation effectiveness of different stages in the second intubation process image. In this disclosure, the stage intubation times, intubation time sequence diagram and intubation ability-time sequence analysis diagram of the first intubation process image are still referred to the above-mentioned FIG. 3A and FIG. 3B as an example, while the stage intubation times, intubation time sequence diagram and intubation ability-time sequence analysis diagram of the second intubation process image are still referred to the above-mentioned FIG. 4A and FIG. 4B as an example.

Referring again to the stage intubation time and intubation time sequence diagram of the first intubation process image as shown in FIG. 3A, the stage from A to B represents the process and time when the Trachway blade of the intubator can correctly enter the oral cavity and reach below the base of tongue (this stage intubation time is about 3 seconds), the stage from B to C presents the process and time to stir up the epiglottis and expose the laryngeal structure (this stage intubation time is about 2.5 seconds), the stage from C to D represents the process and time to adjust the blade and apply appropriate force to expose the optimum glottis structure, and to guide the intubator tube to the larynx (this stage intubation time is about 4 seconds), and the stage from D to E represents the process and time to slide the intubator tube from the larynx into the trachea to positioning (this stage intubation time is about 4.5 seconds). Therefore, the total tracheal intubation time of the stages from A to E (the difference in intubation time between stages) is about 14 seconds. In addition, it can be seen from the stage intubation times and intubation ability-time sequence analysis diagram of the first intubation process image as shown in FIG. 3B that the tracheal intubation ability of the senior anesthesiology attending physician falls between 75% and 100% (i.e., within the gray area as shown in FIG. 3B), which indicates that the intubation skill is quite good.

In the stage intubation time diagram and intubation time sequence diagram of the second (novice) intubation process image as shown in FIG. 4A, the meanings represented by A to E are as described above. In FIG. 4A, the stage intubation time of the stage from A to B is about 6 seconds, the stage intubation time of the stage from B to C is about 19 seconds, the stage intubation time of the stage from C to D is about 30 seconds, and the stage intubation time of the stage from D to E is about 32 seconds, so the total tracheal intubation time of the stages from A to E is about 87 seconds. Comparing FIG. 4A with FIG. 3A, it can be clearly seen that the intubation process of the PGY trainee took a long time, especially the stage from C to D and the stage from D to E. It means that the skill ability of tracheal intubation in the stages from C to E is quite insufficient.

In addition, in the intubation ability-time sequence analysis diagram of the second (novice) intubation process image as shown in FIG. 4B, the intubation images at different times can depict different groups of lines. One group of lines forms a quadrilateral, and each group of lines represents one intubation process. It can also be seen from FIG. 4B that most of these quadrilateral lines fall in the area Z (the gray area in FIG. 4B, which indicates the area where the intubation ability is lower than expected (e.g. the tracheal intubation ability is lower than 75%). This indicates that the initial intubation skill of the PGY intern physician is insufficient and needs to be enhanced. In addition, compared to FIG. 3B, the ability of the stage from the first epiglottis image (Epiglottis 1st) to the first glottis image (Glottis 1st) as shown in FIG. 4B is also insufficient. In other words, the PGY intern experienced intermittent intubation during the intubation process (such as the stage from C to D and the stage from D to E as shown in FIG. 4A), so that it is obvious that the skills of the PGY intern in the stages from C to E are not proficient enough. In addition, the PGY intern also has sporadic cases of insufficient ability at other timing points. Through the above analysis and comparison, in addition to evaluating the tracheal intubation ability of the PGY intern, it can also point out the stage(s) of insufficient ability and the parts where subsequent training needs to be enhanced, thereby achieving the purpose of automatic evaluation of the effectiveness of tracheal intubation.

In summary, it can be seen from the above disclosure that the processing method for tracheal intubation images of the present invention and the effectiveness evaluation method for tracheal intubation including this processing method can correspond the time points at which the recognized objects appear and the time intervals therebetween to the operation times spent in different stages of the tracheal intubation process. It can be used as an evaluation indicator of the effectiveness of tracheal intubation and can also be used as a target for risk factors in the exploration stages. By comparing it with the standardized tracheal intubation time sequences, it will be used to understand the learning effectiveness of novice doctors and provide further detailed learning feedback at each stage. In addition, the present invention can also be used to analyze the discrimination or learning curves when performing intubation with different tools, different levels of personnel, and different difficult intubation scores.

The characteristic of this invention is stated as follow. In the past, the image analysis of tracheal intubation mainly focused on the Cormack grade and failed to identify the influence of other parts. The most important thing is that this invention is to mark multiple anatomical structure characteristics (target objects) to develop the processing system and method for tracheal intubation in time sequence, which can deconstruct the traditional concept that each intubation attempt can only generally succeed or fail as a whole, deconstruct the intubation process into different stages according to the objects and times, and automatically recognize the objects to divide the whole intubation process into different stages and their corresponding spent times, thereby establishing a staged constructed effectiveness evaluation model of tracheal intubation.

This invention can develop artificial intelligent tracheal intubation image processing based on high-risk, high-tech tracheal intubation clinical operations and training needs, and provide real-time time-sequence analysis and objective quantitative evaluation of the entire images. In addition, the present invention also establishes an innovative automatic effectiveness evaluation system for tracheal intubation based on the above-mentioned processing (analysis) method, thereby solving the current problem of subjective judgment and manual interpretation that is difficult to provide immediate auxiliary operation and training feedback. Moreover, the system and method of the invention can achieve the evaluation and development of future tracheal intubation personal learning process techniques, the development and validation of new intubation tools or techniques, and lesson scoring for simulation training.

In summary, the processing method for tracheal intubation images of this invention includes: establishing a database containing a plurality of structural objects, wherein the database stores at least one first intubation process image, and the structural objects are defined by the at least one first intubation process image; performing an object recognition of a second intubation process image based on the defined structural objects, so as to obtain multiple target objects in the second intubation process image identical to the structural objects; and determining multiple time points of recognizing the multiple target objects in the second intubation process image so as to obtain stage intubation times and intubation time sequences of the target objects; wherein the multiple time points of recognizing the multiple target objects in the second intubation process image define n time points, a time difference between any two of the time points of recognizing the target objects is defined as one of the stage intubation times, and a plurality of the stage intubation times are tailored in chronological order to establish the intubation time sequences of the target objects in the second intubation process image. Therefore, based on the clinical operations and training needs of high-risk, high-tech tracheal intubation, the proposed processing method and system for tracheal intubation images of this invention can provide the stage intubation time and intubation time sequence of the entire tracheal intubation image, thereby providing the real-time segmentation of the entire image during staged intubation, serialized processing and analysis of integrated time period, and then establish an effectiveness evaluation for tracheal intubation. As a result, the invention can solve the current problem of subjective judgment and manual interpretation that is difficult to immediately assist operation and training feedback.

The above is only illustrative and not restrictive. Any equivalent modifications or changes without departing from the spirit and scope of the invention shall be included in the appended claims.

What is claimed is:

1. A processing method for tracheal intubation images, comprising:
establishing a database containing a plurality of structural objects, wherein the database stores at least one first intubation process image, and the structural objects are defined by the at least one first intubation process image;
performing an object recognition of a second intubation process image based on the defined structural objects, so as to obtain multiple target objects in the second intubation process image identical to the structural objects; and
determining multiple time points of recognizing the multiple target objects in the second intubation process image so as to obtain stage intubation times and intubation time sequences of the target objects;
wherein, the multiple time points of recognizing the multiple target objects in the second intubation process image define n time points, a time difference between any two of the time points of recognizing the target objects is defined as one of the stage intubation times, and a plurality of the stage intubation times are tailored in chronological order to establish the intubation time sequences of the target objects in the second intubation process image.

2. The processing method of claim 1, wherein the first intubation process image with an intubation difficulty scale of zero is objected to analysis and model establishment so as to define the structural objects.

3. The processing method of claim 1, wherein the structural objects are selected from a group consisting of lip, epiglottis, laryngopharynx, glottis, endotracheal tube, and endotracheal tube marked black line.

4. The processing method of claim 1, further comprising:
determining multiple time points of the structural objects appearing in the first intubation process image so as to obtain stage intubation times and intubation time sequences of the structural objects; wherein the multiple time points of the structural objects appearing in the first intubation process image define n time points, a time difference between any two of the time points of the structural objects appearing in the first intubation process image is defined as one of the stage intubation times, and a plurality of the stage intubation times are tailored in chronological order to establish the intubation time sequences of the structural objects in the first intubation process image; and
drawing an intubation time sequence diagram and an intubation ability-time sequence analysis diagram of the first intubation process image based on the stage intubation times and the intubation time sequences of the structural objects.

5. The processing method of claim 4, further comprising:
drawing an intubation time sequence diagram and an intubation ability-time sequence analysis diagram of the second intubation process image based on the stage intubation times and the intubation time sequences of the target objects.

6. A processing system for tracheal intubation images, comprising:
a database, storing at least one first intubation process image, wherein the at least one first intubation process image defines a plurality of structural objects; and
an electronic device, electrically connected to the database, wherein the electronic device comprises one or more processing units and a memory unit, the one or more processing units are electrically connected to the memory unit, the memory unit stores one or more instructions, and the one or more processing units perform, when executing the one or more instructions, following steps:
performing an object recognition of a second intubation process image based on the defined structural objects, so as to obtain multiple target objects in the second intubation process image identical to the structural objects; and
determining multiple time points of recognizing the multiple target objects in the second intubation process image so as to obtain stage intubation times and intubation time sequences of the target objects;
wherein, the multiple time points of recognizing the multiple target objects in the second intubation process image define n time points, a time difference between any two of the time points of recognizing the target objects is defined as one of the stage intubation times, and a plurality of the stage intubation times are tailored in chronological order to establish the intubation time sequences of the target objects in the second intubation process image.

7. The processing system of claim 6, wherein the database is stored in the memory unit or a cloud device.

8. The processing system of claim 6, wherein the first intubation process image with an intubation difficulty scale of zero is objected to analysis and model establishment so as to define the structural objects.

9. The processing system of claim 6, wherein the structural objects are selected from a group consisting of lip, epiglottis, laryngopharynx, glottis, endotracheal tube, and endotracheal tube marked black line.

10. The processing system of claim 6, wherein the one or more processing units further perform following steps:
determining multiple time points of the structural objects appearing in the first intubation process image so as to obtain stage intubation times and intubation time sequences of the structural objects; wherein the multiple time points of the structural objects appearing in the first intubation process image define n time points, a time difference between any two of the time points of the structural objects appearing in the first intubation process image is defined as one of the stage intubation times, and a plurality of the stage intubation times are tailored in chronological order to establish the intubation time sequences of the structural objects in the first intubation process image; and
drawing an intubation time sequence diagram and an intubation ability-time sequence analysis diagram of the first intubation process image based on the stage intubation times and the intubation time sequences of the structural objects.

11. The processing system of claim 6, wherein the one or more processing units further perform following step:
drawing an intubation time sequence diagram and an intubation ability-time sequence analysis diagram of the second intubation process image based on the stage intubation times and the intubation time sequences of the target objects.

12. An effectiveness evaluation method for tracheal intubation, comprising:
the processing method of claim 5; and
performing an effectiveness evaluation of tracheal intubation with the second intubation process image based on the stage intubation times and the intubation time sequences of the target objects and the structural objects.

13. The evaluation method of claim 12, wherein the effectiveness evaluation of tracheal intubation comprises:
comparing the stage intubation times, the intubation time sequence diagrams and the intubation ability-time sequence analysis diagrams of the first intubation process image and the second intubation process image so as to evaluate intubation effectiveness of different stages in the second intubation process image.
